(19) United States
(12) Patent Application Publication
(10) Pub. No.: US 2008/0021000 A1
(43) Pub. Date: Jan. 24, 2008

(54) MIXTURES OF AND METHODS OF USE FOR POLYUNSATURATED FATTY ACID-CONTAINING PHOSPHOLIPIDS AND ALKYL ETHER PHOSPHOLIPIDS SPECIES

(76) Inventors: Su Chen, Malta, NY (US); Hung Kwong, Malta, NY (US)

Correspondence Address: LAW OFFICE OF MICHAEL A. BLAKE, 112 BROAD STREET, MILFORD, CT, 06460, US

(21) Appl. No.: 11/458,451

(22) Filed: Jul. 19, 2006

(51) Int. Cl. A61K 31/685; C07F 5/06

(52) U.S. Cl. 514/78; 554/76

(57) ABSTRACT

Mixtures of natural phosphatidylcholine species, natural lysophosphatidylcholine species, phosphatidylserine species, phosphatidylethanolamine species, 1-hydroxy-2-acyl-phosphatidylcholine species, 1-hydroxy-2-acyl-phosphatidylserine molecular species, 1-hydroxy-2-acyl-phosphatidylethanolamine molecular species, 1-O-alkyl-2-hydroxy phosphatidylcholine species, 1-O-alkyl-2-docosaheaxnoyl phosphatidylcholine species 1-O-alkyl-2-docosahexaenoyl phosphatidylserine species, and 1-O-alkyl-2-docosahexaenoyl phosphatidylethanolamine species, Methods using the above disclosed mixtures in mammals to treat various conditions.

### FIELD OF THE INVENTION

The present invention relates to (1) the extraction and purification of new mixtures of phosphatidylcholine and alkyl ether phosphatidylcholine species as well as lysophosphatidylcholine species, which are obtained from the liver of saltwater fishes, with the structural characterization of (i) having a mixture of acyl and enriched alkyl fatty chains linked to the sn-1 position of the glycerol backbone, and (ii) having enriched ω-3 polyunsaturated fatty acid chains, in particular docosahexaenoic acid (DHA), linked to the sn-2 position or the sn-1 position of the glycerol backbone; (2) the preparation of disclosed mixtures of phosphatidylcholine, phosphatidylserine and phosphatidylethanolamine species as well as lysophosphatidylcholine, lysophosphatidylserine and lysophosphatidylethanolamine species from the phospholipid species mixtures extracted from the liver of saltwater fishes by enzymatic reactions; and (3) their use as carriers of ω-3-polyunsaturated fatty acids to the brain for the prevention and alleviation of neurodegenerative and neurological diseases which may be caused by the deficiency of ω-3 polyunsaturated fatty acids, in particular DHA.

### BACKGROUND OF THE INVENTION

Omega-3 (or ω-3) polyunsaturated fatty acids, in particular docosahexaenoic acid (DHA), are especially important during prenatal brain development and maintenance of human brain function. Compared with saturated fatty acid, polyunsaturated fatty acid with multiple double bonds within molecule, in particular DHA, causes carbon-carbon chains to become more curved. The more kinked the fatty acid is, the more space it will take up, when it is built into cell membrane phospholipids, in order to keep neuronal membrane functions. This is the main reason that why the brain requires a large amount of nutritionally essential polyunsaturated fatty acids, especially DHA, because DHA and DHA-containing molecular species of phospholipids may contribute to important brain functions including signal transduction and information processing [Akbar et al., Docosahexaenoic acid: a positive modulator of Akt signaling in neuronal survival. Proc. Natl. Acad. Sci. U.S.A. 102: 10858 (2005)]. Alteration of neuronal membrane DHA-containing phospholipid species can not only influence crucial intracellular and intercellular signaling but also alter many membrane physical properties such as fluidity, phase transition temperature and bilayer thickness. The deficiency of DHA markedly affects neurotransmission, membrane-bound enzyme and ion channel activities leading to brain aging, Alzheimer's disease, Parkinson's disease, schizophrenia and peroxisomal disorders. For example, a study indicated that the concentration of DHA in patients with Alzheimer's disease is significantly decreased [Conquer, et al., Fatty acid analysis of blood plasma of patients with Alzheimer's disease, other type of dementia, and cognitive impairment, Lipids, 35:1305 (2000)]. The studies of Garcia et al. [Garcia et al., Effect of docosahexaenoic acid on the synthesis of phosphatidylserine in rat brain in misrosomes and C6 gliome cells. J. Neurochem. 70: 24 (1998); Kim et al., Inhibition of neuronal apoptosis by docosahexaenoic acid (22:6n-3): Role of phosphatidylserine in anti-apoptotic effect. J. Biol. Chem. 275:35215 (2000)] found out the new role of DHA and phosphatidylserine in neuronal apoptosis, indicating that exogenous DHA may enhance phosphatidylserine accumulation in apoptotic Neuro-2A cells leading to the protection of neuronal cells from apoptotic death. The studies strongly suggest that one of supporting roles for anti-apoptosis of neurons is supplying DHA to the brain.

Because the human body cannot synthesize ω-3 polyunsaturated fatty acids, in particular DHA, exogenous introduction of DHA to human has been applied. There are a few products available for use as brain nutrients, such as fish oils (DHA-containing neutral lipids) and similar products.

Although these products contain DHA and other omega-3 polyunsaturated fatty acids, experiments have demonstrated that only a very small amount of DHA can be found in the brain after administering a large amount of these products. But an early study showed that DHA-containing lysophospholipid in albumin, rather than the forms of free DHA and other esterified DHA, is preferred in the uptake of DHA in the brain of young rats when an in vitro model of blood-brain barrier is used [Thies et al., Unsaturated fatty acids esterified in 2-acyl-1-lysophosphatidylcholine bound to albumin are more efficiently taken up by the young rat brain than unesterified form. J. Neurochem. 59: 1110 (1992)].

Interestingly, a study reported that dietary phospholipid with DHA-containing molecular species as supplementation is much more efficient than soybean phospholipid for ensuring a normal level DHA in the brain during the period of brain development in rats [Bourre and Dumont, Neurosci. Lett., 335:129 (2002)] because DHA species are absent in the latter. The result suggests that DHA-containing phospholipid species are effective forms to be used as DHA carriers to brain.

Phosphatidylcholine (PC), phosphatidylserine (PS) and phosphatidylethanolamine (PE) as well as lysophosphatidylcholine (Lyso PC), lysophosphatidylserine (Lyso PS) and lysophosphatidylethanolamine (Lyso PE) are naturally occurring phospholipid classes, existing in mixture forms of the molecular species. The structural diversity of the molecular species of phospholipids has been described in detail [Chen, Lipids, 28, 85 (1997); Chen et, al. Biomed. Mass Spectrom. 21, 655 (1992)]. Biochemical and biophysical functions of phospholipids are well documented and appear to be determined by the fatty acid composition of the lipids.

Ether phospholipids are usually found in animal tissues and human cells as minor components, existing together with molecular species of diacyl phospholipids carrying the same polar head group. It is well known that there are two predominant types of ether bonds in the phospholipid. One form is represented by the plasmalogens (with 1-alk-1′-enyl fatty chain linked to the sn-1 position of the glycerol backbone), which is the most abundant subclass of phospholipids in most tissues. The other form is alkyl phospholipids that contain 1-O-alkyl fatty chain(s) linked to the sn-1 position of the glycerol backbone. Although mixtures of phospholipids and ether phospholipids have been found in animals and humans [Diagne, et. al., Studies on ether phospholipids, Biochim. Biophys. Acta, 793, 221 (1984)], little has been described regarding the presence of phospholipid species mixtures containing 1-O-alkyl-2-DHA molecules, as well as DHA-containing lysophospholipids in aquatic animals.

Investigation of PC metabolism in human [Galli et al., Prolonged retention of doubly labeled phosphatidylcholine in human plasma and erythrocytes after oral administration. Lipids, 27: (1992)] indicated that a major portion of PC species can be found as intact molecules in plasma after oral administration of labeled phospholipid species. The result is supported by an animal experiment, suggesting that more than 80% of PC, which is recovered from the intestinal lymph of rats, is still intact after oral administration of phospholipids [Ikeda et al., Absorption and transport of base moieties of phosphatidylcholine and phosphatidylethanolamine in rats, Biochim. Biophys. Acta, 921; 245 (1987)].

However, fatty acid chains of phospholipids can be further hydrolyzed by lecithin-cholesterol acyltransferase in plasma after molecular species are incorporated into high density lipoprotein, and followed by phospholipase A₁ and phospholipase A₂ hydrolyses in the liver. Recently, several papers reported the possibility of transporting high density lipoprotein across the blood-brain barrier using an in vitro model [Balazs et al., Uptake and transport of high-density lipoprotein (HDL) and HDL-associated α-tocopherol by an in vitro blood-brain barrier model. J. Neurochem. 89: 939 (2004)] and the importance of endothelial lipase in the metabolism of high density lipoprotein associated phospholipids at the blood-brain barrier [Ma et al., Endothelial lipase is a major genetic determinant for high-density lipoprotein concentration, structure, and metabolism. Proc. Natl. Acad. Sci. U.S.A. 100: 2748 (2003)], demonstrating that endothelial lipase (i) exhibits primarily the activity of phospholipase A₁ leading to the hydrolysis of sn-1 fatty acid chains from the molecular species of high density lipoprotein phospholipids, and (ii) is inactive in the hydrolysis of ether phospholipid molecular species [Gauster et al., Endothelial lipase release saturated and unsaturated fatty acid of high density lipoprotein phosphatidylcholine. J. Lipid Res. 46: 1517 (2005)].

An early study also showed that after the administration of lysophospholipids, these lipid species do not enhance fatty acid chain retention in mucosa and may survive from the hydrolysis of lecithin-cholesterol-acyltransferase in plasma, as well as other phospholipases in the liver [Viola et al., Absorption and distribution of arachidonate in rats receiving lysophospholipids by oral route, J. Lipid Res. 34, 1843 (1993)].

Based on these published results, it is clear to see that ether phospholipid species, such as 1-O-alkyl-2-acyl molecular species, and lysophospholipid molecular species are more stable in vivo lipid metabolism, compared with related acyl species. Because ether phospholipid and lysophospholipid species can be survived from blocking due to the hydrolysis of phospholipase A₁ and phospholipase A₁-like enzymes in vivo metabolism [Shamburek et al., Disappearance of two major phosphatidylcholine from plasma is predominantly via LCAT and hepatic lipase, Am J. Physiol. 271: E1073 (1996); plasma 1-palmitoyl-2-linoleoyl phosphatidylcholine. Evidence for extensive phospholipase A₁ hydrolysis and hepatic metabolism of the products, J. Biol. Chem. 266: 18002 (1991)], DHA-containing ether phospholipid species and DHA-containing lysophospholipid species can be delivered smoothly into the brain as carriers of DHA, resulting in the uptake of free DHA after brain phospholipase hydrolyses of the phospholipids species [Ross et al., Characterization of a novel phospholipase A₂ activity in human brain. J. Neurochem. 64, 2213 (1995)].

Interest is focused on the preparation of phospholipid species containing 1-O-alkyl-2-DHA molecules and DHA-containing lysophospholipid species. However, obtaining molecular species mixtures containing enriched DHA-containing ether phospholipids and -lysophospholipids species from aquatic animals is poorly understood [Chapelle, Plasmalogen and O-alkyl glycerophospholipids in aquatic animals, Comp. Biochem. Physiol. 88, 1 (1987)]. Additionally, the chemical synthesis of these phospholipids species is both difficult and expensive, and thus a large-scale preparation of the molecular species for use as potential brain health supplementation is not available. Thus there is a need for a method of a large-scale preparation of DHA-containing phospholipids and -lysophospholipids species for use as potential brain health supplementations.

### SUMMARY OF THE INVENTION

The disclosed invention relates to a mixture of natural phosphatidylcholine species as shown in Formula 1:

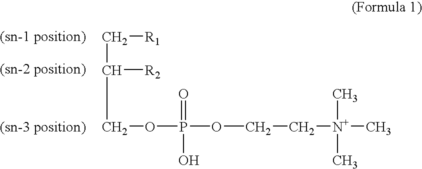

where R1 is a mixture of acyl and alkyl fatty chains, linked to the sn-1 position, selected from the group consisting of COOC₁₅H₃₁ (acyl fatty chain; palmitic acid; 16:0); COOC₁₆H₃₃ (acyl fatty chain; margaric acid; 17:0); COOC₁₇H₃₅ (acyl fatty chain; stearic acid; 18:0); OC₁₆H₃₃ (alkyl fatty chain; O-16:0); OC₁₈H₃₇ (alkyl fatty chain; O-18:0); and OC₁₈H₃₅ (alkyl fatty chain; O-18:1); and

where R₂ consists of a mixture of acyl fatty chains, linked to the sn-2 position, and selected from the group consisting of COOC₁₃H₂₇ (acyl fatty chain; myristic acid; 14:0); COOC₁₅H₃₁ (acyl fatty chain; palmitic acid; 16:0); COOC₁₅H₂₉ (acyl fatty chain; palmitoleic acid; 16:1); COOC₁₇H₃₃ (acyl fatty chain; oleic acid; 18:1); COOC₁₉H₃₁ (acyl fatty chain; arachidonic acid; 20:4); COOC₁₉H₂₉ (acyl fatty chain; eicosapentaenoic acid; 20:5 (ω-3)); COOC₂₁H₃₃ (acyl fatty chain; docosapentanoic acid; 22:5); and COOC₂₁H₃₁ (acyl fatty chain; docosahexaenoic acid; 22:6 (ω-3)).

The disclosed invention also relates to a mixture of natural lysophosphatidylcholine species as shown in Formula 2:

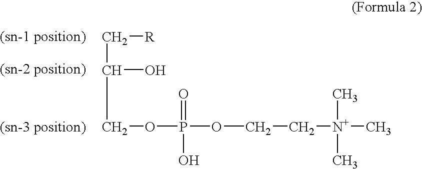

where R is a mixture of acyl and alkyl fatty chains, linked to the sn-1 position, and selected from the group consisting of COOC₁₃H₂₇ (acyl fatty chain; myristic acid; 14:0); COOC₁₅H₃₁ (acyl fatty chain; palmitic acid; 16:0); COOC₁₅H₂₉ (acyl fatty chain; palmitoleic acid; 16:1); COOC₁₇H₃₅ (acyl fatty chain; stearic acid; 18:0); COOC₁₇H₃₃ (acyl fatty chain; oleic acid; 18:1); COOC₁₇H₃₁ (acyl fatty chain; linoleic acid; 18:2); COOC₁₉H₃₁ (acyl fatty chain; arachidonic acid; 20:4); COOC₁₉H₂₉ (acyl fatty chain; eicosapentaenoic acid; 20:5 (ω-3)); COOC₂₁H₃₁ (acyl fatty chain; docosahexaenoic acid; 22:6 (ω-3)); OC₁₄H₂₉ (alkyl fatty chain; O-14:0); OC₁₆H₃₃ (alkyl fatty chain; O-16:0); OC₁₆H₃₁ (alkyl fatty chain; O-16:1); OC₁₈H₃₇ (alkyl fatty chain; O-18:0); and OC₁₈H₃₅ (alkyl fatty chain; O-18:1).

The disclosed invention additionally, relates to a mixture of phosphatidylserine species as shown in Formula 3:

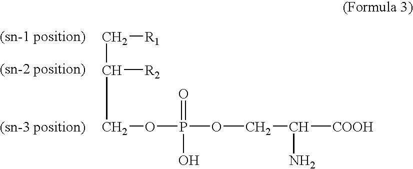

where R₁ is a mixture of acyl and alkyl fatty chains, linked to the sn-1 position, selected from the group consisting of COOC₁₅H₃₁ (acyl fatty chain; palmitic acid; 16:0); COOC₁₆H₃₃ (acyl fatty chain; margaric acid; 17:0); COOC₁₇H₃₅ (acyl fatty chain; stearic acid; 18:0); OC₁₆H₃₃ (alkyl fatty chain; O-16:0); OC₁₈H₃₇ (alkyl fatty chain; O-18:0); and OC₁₈H₃₅ (alkyl fatty chain; O-18:1); and

where R₂ consists of a mixture of acyl fatty chains, linked to the sn-2 position, and selected from the group consisting of COOC₁₃H₂₇ (acyl fatty chain; myristic acid; 14:0); COOC₁₅H₃₁ (acyl fatty chain; palmitic acid; 16:0); COOC₁₅H₂₉ (acyl fatty chain; palmitoleic acid; 16:1); COOC₁₇H₃₃ (acyl fatty chain; oleic acid; 18:1); COOC₁₉H₃₁ (acyl fatty chain; arachidonic acid; 20:4); COOC₁₉H₂₉ (acyl fatty chain; eicosapentaenoic acid; 20:5 (ω-3)); COOC₂₁H₃₃ (acyl fatty chain; docosapentanoic acid; 22:5); and COOC₂₁H₃₁ (acyl fatty chain; docosahexaenoic acid; 22:6 (ω-3)).

The disclosed invention, also, relates to a mixture of phosphatidylethanolamine species as shown in Formula 4:

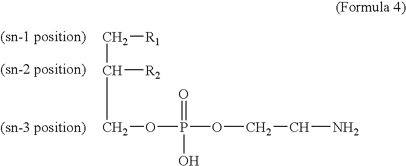

where R₁ is a mixture of acyl and alkyl fatty chains, linked to the sn-1 position, selected from the group consisting of COOC₁₅H₃₁ (acyl fatty chain; palmitic acid; 16:0); COOC₁₆H₃₃ (acyl fatty chain; margaric acid; 17:0); COOC₁₇H₃₅ (acyl fatty chain; stearic acid; 18:0); OC₁₆H₃₃ (alkyl fatty chain; O-16:0); OC₁₈H₃₇ (alkyl fatty chain; O-18:0); and OC₁₈H₃₅ (alkyl fatty chain; O-18:1); and

where R₂ consists of a mixture of acyl fatty chains, linked to the sn-2 position, and selected from the group consisting of COOC₁₃H₂₇ (acyl fatty chain; myristic acid; 14:0); COOC₁₅H₃₁ (acyl fatty chain; palmitic acid; 16:0); COOC₁₅H₂₉ (acyl fatty chain; palmitoleic acid; 16:1); COOC₁₇H₃₃ (acyl fatty chain; oleic acid; 18:1); COOC₁₉H₃₁ (acyl fatty chain; arachidonic acid; 20:4); COOC₁₉H₂₉ (acyl fatty chain; eicosapentaenoic acid; 20:5 (ω-3)); COOC₂₁H₃₃ (acyl fatty chain; docosapentanoic acid; 22:5); and COOC₂₁H₃₁ (acyl fatty chain; docosahexaenoic acid; 22:6 (ω-3)).

In addition, the disclosed invention relates to a mixture of 1-hydroxy-2-acyl-phosphatidylcholine species as shown in Formula 5.

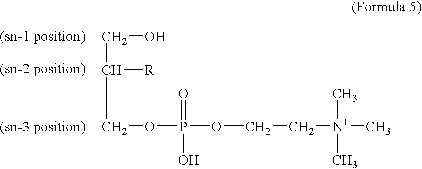

wherein R is a mixture of acyl fatty chains linked to the sn-2 position, selected from the group consisting of COOC₁₅H₃₁ (acyl fatty chain; palmitic acid; 16:0); COOC₁₇H₃₃ (acyl fatty chain; oleic acid; 18:1); COOC₁₉H₃₁ (acyl fatty chain; arachidonic acid; 20:4); COOC₁₉H₂₉ (acyl fatty chain; eicosapentaenoic acid; 20:5 (ω-3)); COOC₂₁H₃₃ (acyl fatty chain; docosapentanoic acid; 22:5); and COOC₂₁H₃₁ (acyl fatty chain; docosahexaenoic acid; 22:6 (ω-3)).

Additionally, the disclosed invention relates to a mixture of 1-hydroxy-2-acyl-phosphatidylserine molecular species as shown in Formula 6:

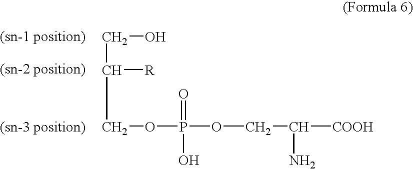

wherein R is a mixture of acyl fatty chains linked to the sn-2 position, selected from the group consisting of COOC₁₅H₃₁ (acyl fatty chain; palmitic acid; 16:0); COOC₁₇H₃₃ (acyl fatty chain; oleic acid; 18:1); COOC₁₉H₃₁ (acyl fatty chain; arachidonic acid; 20:4); COOC₁₉H₂₉ (acyl fatty chain; eicosapentaenoic acid; 20:5 (ω-3)); COOC₂₁H₃₃ (acyl fatty chain; docosapentanoic acid; 22:5); and COOC₂₁H₃₁ (acyl fatty chain; docosahexaenoic acid; 22:6 (ω-3)).

Furthermore, the disclosed invention relates to a mixture of 1-hydroxy-2-acyl-phosphatidylethanolamine molecular species as shown in Formula 7:

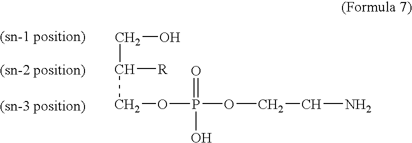

wherein R is a mixture of acyl fatty chains linked to the sn-2 position, selected from the group consisting of COOC₁₅H₃₁ (acyl fatty chain; palmitic acid; 16:0); COOC₁₇H₃₃ (acyl fatty chain; oleic acid; 18:1); COOC₁₉H₃₁ (acyl fatty chain; arachidonic acid; 20:4); COOC₁₉H₂₉ (acyl fatty chain; eicosapentaenoic acid; 20:5 (ω-3)); COOC₂₁H₃₃ (acyl fatty chain; docosapentanoic acid; 22:5); and COOC₂₁H₃₁ (acyl fatty chain; docosahexaenoic acid; 22:6 (ω-3)).

Also, the disclosed invention relates to a mixture of 1-O-alkyl-2-hydroxy phosphatidylcholine species as Formula 8:

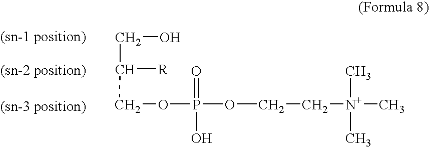

wherein R is a mixture of alkyl fatty chains linked to the sn-1 position, selected from the group consisting of OC₁₄H₂₉ (alkyl fatty chain; O-14:0); OC₁₆H₃₃ (alkyl fatty chain; O-16:0); OC₁₆H₃₁ (alkyl fatty chain; O-16:1); OC₁₈H₃₇ (alkyl fatty chain; O-18:0); and OC₁₈H₃₅ (alkyl fatty chain; O-18:1).

The disclosed invention, in addition, relates to a mixture of 1-O-alkyl-2-docosaheaxnoyl phosphatidylcholine species as shown in Formula 9:

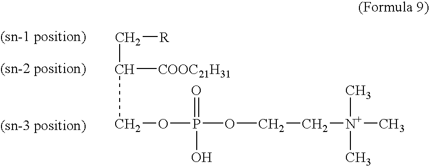

where R is a mixture of alkyl fatty chains linked to the sn-1 position, selected from the group consisting of OC₁₄H₂₉ (alkyl fatty chain; O-14:0); OC₁₆H₃₃ (alkyl fatty chain; O-16:0); OC₁₆H₃₁ (alkyl fatty chain; O-16:1); OC₁₈H₃₇ (alkyl fatty chain; O-18:0); and OC₁₈H₃₅ (alkyl fatty chain; O-18:1).

In addition, the disclosed invention relates to a mixture of 1-O-alkyl-2-docosahexaenoyl phosphatidylserine species as shown in Formula 10:

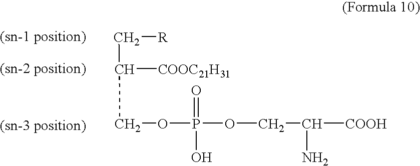

where R is a mixture of alkyl fatty chains linked to the sn-1 position, selected from the group consisting of OC₁₄H₂₉ (alkyl fatty chain; O-14:0); OC₁₆H₃₃ (alkyl fatty chain; O-16:0); OC₁₆H₃₁ (alkyl fatty chain; O-16:1); OC₁₈H₃₇ (alkyl fatty chain; O-18:0); and OC₁₈H₃₅ (alkyl fatty chain; O-18:1).

Furthermore, the disclosed invention relates to a mixture of 1-O-alkyl-2-docosahexaenoyl phosphatidylethanolamine species as shown in Formula 11.

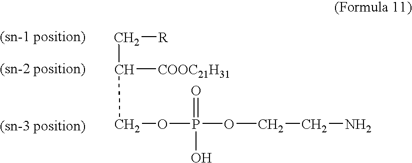

where R is a mixture of alkyl fatty chains linked to the sn-1 position, selected from the group consisting of OC₁₄H₂₉ (alkyl fatty chain; O-14:0); OC₁₆H₃₃ (alkyl fatty chain; O-16:0); OC₁₆H₃₁ (alkyl fatty chain; O-16:1); OC₁₈H₃₇ (alkyl fatty chain; O-18:0); and OC₁₈H₃₅ (alkyl fatty chain; O-18:1).

### In addition, the disclosed invention relates to a method of using the above disclosed mixtures in mammals to treat various conditions.

### DETAILED DESCRIPTION OF THE INVENTION

Surprisingly, the applicants have discovered that it is possible to obtain PC species mixtures with enriched 1-O-alkyl-2-DHA molecules and Lyso PC species mixtures with enriched DHA-containing molecule from the liver of saltwater fishes by chemical extraction, followed by column purification. For example, the PC species mixture from common dolphin liver contains approximately 50% of sn-2-(ω-3)-polyunsaturated fatty acid molecules, including approximately 25% of 1-O-alkyl-2-DHA molecules; for another example, the Lyso PC species mixture from shark liver contains approximately 33% of DHA-containing molecule. The collection of raw liver materials in a large quantity is readily available from marine animals.

A second aspect of the present invention is that it is possible to prepare disclosed mixtures of PS and PE species by the transphosphatidylation [Comfurius et, al. Enzymatic synthesis of phosphatidylserine on small scale by use of a one-phase system, J. Lipid Res. 31, 1719 (1990)] using the special starting material combinations that consist of (i) phospholipase D, (ii) L-serine or ethanolamine and (iii) the PC species mixtures extracted from the liver of saltwater fishes.

A third aspect of the present invention relates to the preparation of disclosed sn-2-DHA-Lyso PC, -Lyso PS and -Lyso PE species mixtures by lipase hydrolyses of mixtures of the liver PC species, as well as transphosphatidylated fish liver-PS and -PE species [Slotboom et al., Hydrolysis of phosphoglycerides by Purified lipase preparations II. Preparation of unsaturated 2-monoacyl choline phosphoglycerides, Chem. Phys. Lipids, 4, 30 (1970)].

A fourth aspect of the present invention is involved in the preparation of disclosed mixtures of 1-O-alkyl-2-DHA-PC, -PS and -PE species using 1-O-alkyl-2-hydroxy-PC species mixtures [Paltauf et al., In Method in Enzymology, 197, 141 (1991)] as the starting material, which are prepared from the liver PC species and/or the liver Lyso PC species mixtures.

Compared with available phospholipid product Lecithin that contains soybean PC species mixtures, newly discovered and purified PC and Lyso PC species mixtures are alternative supplementations. Furthermore, the PC and Lyso PC may be used as brain nutrients for the delay of brain aging. However, Lecithin containing PC species mixtures lack DHA molecular species.

A mixture of bovine cortex phosphatidylserine (BCPS) species, which contains approximately 8% of sn-2-DHA molecular species, was registered as a drug in Europe for both cognitive deficits in the old and the alleviation of Alzheimer's disease [Crook et al., Effect of phosphatidyl-serine in Alzheimer's Disease, Psychopharmacology Bulletin 28, 61(1992); Pepeu, et al., A review of phosphatidylserine pharmaceutical and clinical effects: Is phosphatidylserine a drug for aging brain? Pharmacol. Res. 33, 51 (1996)]. Unfortunately, the risk of bovine spongiform encephalopathy made the use of the BCPS extracted from bovine brain potentially dangerous.

Compared with the BCPS, disclosed mixtures of transphosphatidylated fish liver-PS and -PE species, as well as sn-2-DHA-Lyso PS and -Lyso PE and alkyl-DHA-PC, -PS and -PE species contain much more sn-2-DHA molecular species (over 40%, even 100%), and are considered to be of much more therapeutic interest in the alleviation and treatment of neurodegenerative and neurological diseases, which are associated with dysfunctions of neurotrophins and neurotrophin receptors caused by the deficiency of (ω-3) polyunsaturated fatty acids, in particular DHA.

Compared with commercially available phospholipids product transphosphatidylated soybean PS species mixtures, disclosed phospholipid species mixtures are qualified for use as substitutes of the BCPS for the treatment of neurodegenerative and neurological diseases caused by DHA deficiency, because DHA-containing phospholipid species are absent in currently available transphosphatidylated soybean PS species mixtures (http://www.cfsan.fda.gov/˜dms/dsltr36.html).

Although the preparation of a mixture of transphosphatidylated fish brain-PS species has been reported (European patent: EPO 819 760 A1), the disadvantages of the preparation include: (i) the collection of fish brain material in a large quantity is difficult, and thus the product preparation in industrial scale is limited; (ii) Lyso PC has less than 5% of fish brain phospholipids, and DHA-containing Lyso PC is almost absent; and (iii) 1-O-alkyl-DHA phospholipids species are almost absent in fish brain.

Although the preparation of a mixture of transphosphatidylated fish skin-PS has been also reported, [Hosokawa et al., Conversion to docosahexaenoic acid-containing phosphatidylserine from squid skin lecithin by phospholipase D-mediated transphosphatidylation, J. Agric. Food. Chem. 48, 4550 (2000)], the collection of fish skin as starting materials in a large quantity is much more difficult, and thus an industrial scale preparation of the product is almost impossible. Furthermore, 1-O-alkyl-DHA phospholipids species and DHA-containing lysophospholipids are almost absent in fish skin as well.

Materials and procedures described in this disclosure have made it possible to prepare a series of disclosed mixtures of phospholipid species in a large scale, and even in an industrial scale. The resulting products can be used as the preventive and therapeutic reagents for brain and mental health.

### EXAMPLE 1

### Preparation of Crude Lipids Extracted from the Liver of Saltwater Fishes

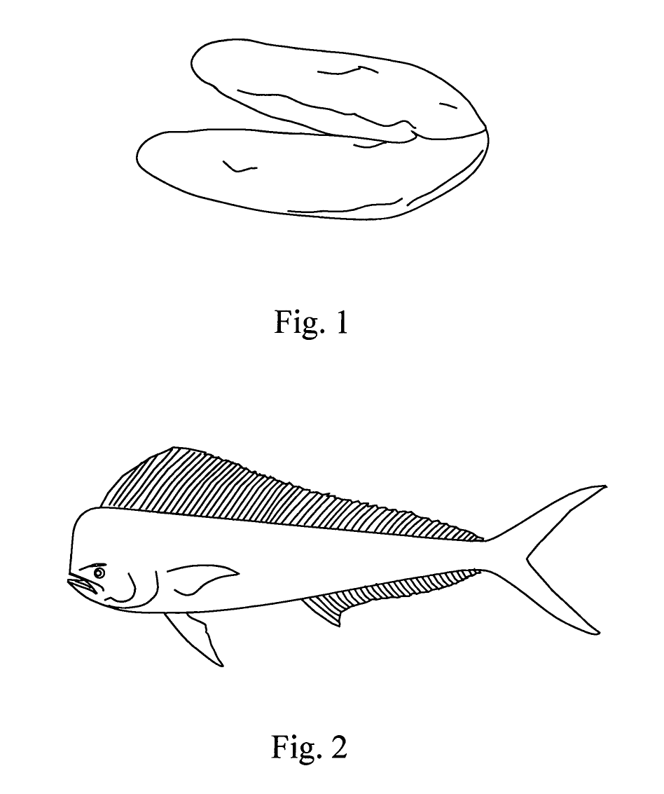

About two sharks were obtained in a fish market near the city of Medan in Indonesia, and the livers (approximately 1,170 grams (FIG. 1; see below) and 550 grams of the wet liver material, respectively) were transported in ice and then passed through a hand meat grinder. Tiny liver fragments were made with an electronic blender and were then mixed with about 20 volumes of cold acetone standing for four hours. After removing the acetone, the liver fragments, now the pellets were dried under nitrogen, and then the dried pellets were homogenized with about 10 volumes (w/v) of ethanol/ethyl acetate (1/1; v/v) stirring overnight. After evaporation of the ethanol/ethyl acetate, about 172 grams of crude lipids extracted from 1,170 grams of the wet liver material were obtained.

About 50 grams of the crude lipids were mixed with about 10 volumes (w/v) of acetone, and then the solution was stirred at about 30° C. for about 90 min. The mixture was filtered, and the acetone extract was kept at about −20° C. for overnight. The mixture solution was rapidly filtered and led to the precipitation of crude phospholipids (approximately 23.4 grams), consisting of about 80% of PC, about 15% of Lyso PC and about 5% of PE and others. The crude lipids and phospholipids are also harvested from the livers of frozen common dolphin (FIG. 2; about 770 grams of the wet liver material), tuna (about 720 grams of the wet liver material) and salmon (about 810 grams of the wet liver material), which were provided by a Fishing Company located in the province of Fujian in China, using the procedure described above.

### EXAMPLE 2

### Purification of PC and Lyso PC from Crude Phospholipids

Mixtures of PC and Lyso PC species were purified from about 10 grams of crude phospholipids by silica gel chromatography using an axial pressure in an about 250 mL-column equilibrated with chloroform/methanol (95/5); gradient elution was performed with mixtures of chloroform/methanol 90/10 (v/v), 80/20 (v/v), 70/30 (v/v), and 60/40 (v/v). PC species mixture was eluted from the column with a mixture of chloroform/methanol/water 50/50/1 (v/v/v), monitored by thin layer chromatography (TLC); and Lyso PC species mixture was eluted from the column with a mixture of chloroform/methanol/water 50/50/5 (v/v/v), monitored by TLC. The structures and percentage of purified PC and Lyso PC species mixtures, which were obtained by intensities of their protonated molecules of mass spectrometry [Brugger et al., Proc. Natl. Acad. Sci. USA, 94, 2339 (1997); Liebisch et al., Clin. Chem. 48, 2217 (2002)], are shown below.

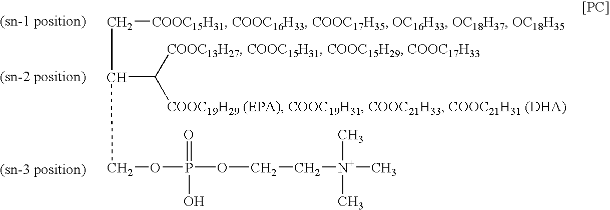

| [M+H]⁺ | Molecular Species* | Dolphin (%) | Shark(%) | Tuna (%) | Salmon (%) |
|---|---|---|---|---|---|
| 834.6 | 18:0–22:6 | 2.6 ± 0.5** | 7.4 ± 2.0 | 7.7 ± 1.1 | 4.7 ± 0.2 |
| 822.6 | aa18:0–22:5*** | 3.4 ± 0.3 | 2.8 ± 0.3 | 2.9 ± 0.7 | 2.7 ± 0.6 |
| 820.6 | 17:0–22:6 | 9.6 ± 1.1 | 7.8 ± 2.3 | 3.9 ± 0.3 | 4.7 ± 0.4 |
| 818.6 | aa18:1–22:6 | 7.2 ± 1.7 | 6.9 ± 1.7 | 3.6 ± 0.7 | 2.3 ± 0.2 |
| 806.6 | 16:0–22:6 | 12.6 ± 2.2 | 15.9 ± 3.3 | 19.7 ± 2.7 | 13.9 ± 2.3 |
| 794.6 | aa18:1–20:4 | 3.5 ± 0.7 | 3.9 ± 1.1 | 2.7 ± 0.5 | 3.0 ± 0.8 |
| | aa16:0–22:5 | | | | |
| 792.6 | aa16:0–22:6 | 17.3 ± 2.1 | 9.8 ± 1.2 | 4.2 ± 1.2 | 12.5 ± 1.7 |
| | aa18:1–20:5 | | | | |
| 782.6 | 16:0–20:4 | 4.0 ± 0.6 | 4.8 ± 1.1 | 7.3 ± 0.3 | 4.8 ± 1.1 |
| 780.6 | 16:0–20:5 | 5.9 ± 1.1 | 2.8 ± 0.5 | 4.2 ± 0.8 | 7.0 ± 1.6 |
| 760.6 | 16:0–18:1 | 9.4 ± 2.0 | 7.8 ± 1.0 | 12.1 ± 2.5 | 11.2 ± 2.1 |
| 746.6 | aa16:0–18:1 | 2.5 ± 0.7 | 9.9 ± 1.6 | 6.3 ± 1.1 | 3.3 ± 0.3 |
| 734.6 | 16:0–16:0 | 11 ± 1.7 | 9.2 ± 1.8 | 12.8 ± 1.9 | 13.9 ± 3.1 |
| 732.6 | 16:0–16:1 | 3.1 ± 0.6 | 3.0 ± 1.7 | 3.2 ± 0.5 | 3.7 ± 1.1 |
| 720.6 | aa16:0–16:0 | 3.2 ± 1.1 | 2.6 ± 0.2 | 3.9 ± 0.8 | 3.4 ± 0.9 |
| 718.6 | aa16:0–16:1 | 1.2 ± 0.3 | 2.1 ± 1.1 | 2.6 ± 0.4 | 1.4 ± 0.7 |
| 706.6 | 16:0–14:0 | 3.3 ± 1.5 | 3.2 ± 1.3 | 3.6 ± 1.1 | 7.6 ± 1.4 |

| | | | | |
|---|---|---|---|---|
| Total ω-3 fatty acid species | 55.2 | 50.6 | 43.3 | 45 |
| Total DHA species | 49.3 | 47.8 | 39 | 38 |
| Total acylalkyl DHA species | 24.5 | 16.7 | 7.8 | 14.8 |
| Total acylalkyl species | 38.3 | 37.9 | 26 | 28.6 |

*X:Y–x:y (For example 16:0–22:6), where X is the total carbon number of fatty acids esterified at sn − 1 position, and Y is the total unsaturation degrees of the fatty acid groups; where x is the total carbon number of fatty acids esterified at sn-2 position, and y is the total unsaturation degrees of the fatty acid groups (Chen, Lipids, 35, 85 (1997)).
**Semiquantitative results were generated from the intensity average of protonated molecules ([M + H]⁺) of the molecular species of the liver PCs, based on three replicate experiments of mass spectrometry.
***aa16:0–22:5 is alkylacyl PC species

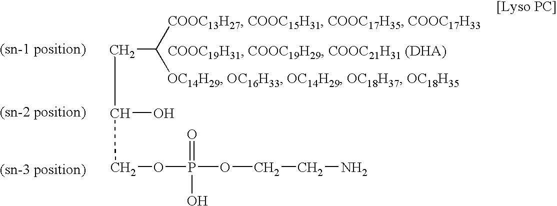

| | Molecular | | | |
|---|---|---|---|---|
| [M + H]⁺ | Species | Dolphin (%) | Shark(%) | Tuna (%) |
| 568.5 | 22:6 | 31.1 ± 1.3 | 33.3 ± 1.6 | 23.9 ± 1.4 |
| 544.5 | 20:4 | 7.8 ± 0.3 | 8.5 ± 0.9 | 9.5 ± 0.1 |
| 542.5 | 20:5 | 8.1 ± 0.7 | 8.0 ± 0.7 | 3.4 ± 0.3 |
| 524.5 | 18:0 | 6.4 ± 0.3 | 6.8 ± 0.7 | 13.8 ± 0.3 |
| 522.5 | 18:1 | 12.5 ± 0.6 | 11.9 ± 0.8 | 7.2 ± 0.3 |
| 510.5 | a18:0* | 2.0 ± 0.4 | 2.1 ± 0.5 | 3.6 ± 0.2 |
| 508.5 | a18:1 | 3.0 ± 0.2 | 3.1 ± 0.3 | 2.5 ± 0.2 |
| 496.5 | 16:0 | 23.3 ± 1.7 | 21.4 ± 1.5 | 29.4 ± 2.5 |
| 482.5 | a16:0 | 2.3 ± 0.2 | 1.9 ± 0.8 | 3.5 ± 0.4 |
| 480.5 | a16:1 | 0.4 ± 0.04 | 1.3 ± 0.1 | 1.2 ± 0.3 |
| 468.5 | 14:0 | 2.8 ± 0.2 | 2.5 ± 0.8 | 1.7 ± 0.7 |
| 454.5 | a14:0 | 0.3 ± 0.1 | 1.3 ± 0.06 | 0.5 ± 0.3 |

| | | | |
|---|---|---|---|
| Total Alkyl species | 7.7 | 7.6 | 10.6 |
| Total DHA species | 31.1 | 33.3 | 23.9 |

*a18:0 is alkyl chain at the sn-1 position

### EXAMPLE 3

### Preparation of Mixtures of Transphosphatidylated-Fish Liver PS Species

About 4 mL of acetate buffer (0.2M; pH 5.5), containing about 40 mM of calcium chloride and two grams of L-serine, were prepared at about 45° C. and then placed into an 20-mL disposable vial containing about 400 mg of purified PC from shark liver or about 450 mg of crude phospholipids. The vial was generally fully filled with argon. The enzymatic reaction was started by adding about 10 Units of phospholipase D (Streptomyces sp, BIOMOL, Plymouth Meeting, Pa., U.S.A). The mixture was left to react for about 15 hours at about 45° C. on a shaker. Once the reaction was complete, the vial was unloaded with about 40 mL of a mixture of chloroform/methanol 2/1 (v/v) and about 4 mL of water. The two separated phases were formed, and PS was in the down-phase. PS remaining in the up-phase was re-extracted with chloroform. After drying the combined two organic phases, PS was purified by silica chromatography, as described in Example 2, with an about 250 mL-column. PS was eluted with a mixture of chloroform/methanol 55/45 (v/v). About 285 mg of the product were obtained after purification. PS species mixture was also made from the PC species mixture of common dolphin liver, as described above. Structures of the PS products are shown below. The molecular species were identified by negative-ion electrospray mass spectrometry.

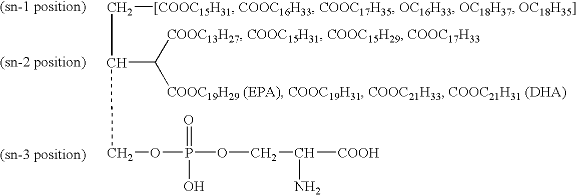

### EXAMPLE 4

### Preparation of Mixtures of Transphosphatidylated-Fish Liver PE Species

About 4 mL of acetate buffer (0.2M; pH 5.5), containing about 40 mM of calcium chloride and about 800 mg of ethanolamine were prepared in an about 20-mL disposable vial at room temperature, and then the pH of the solution was adjusted to about pH 5.5 with glacial acetic acid. About 400 mg of purified PC from shark liver or about 450 mg of crude phospholipids were added. The vial was generally fully filled with argon. The enzymatic reaction was started by adding about 10 Units of phospholipase D (Streptomyces sp, BIOMOL, Plymouth Meeting, Pa., USA). The mixture was left to react for about 15 hours at about 45° C. on a shaker. Once the reaction was complete, the vial was unloaded with about 40 mL of a mixture of chloroform/methanol 2/1 (v/v) and about 4 mL of water. The two separated phases were formed, and PE was in the down-phase. PE remaining in the up-phase was re-extracted with chloroform. After drying the combined two organic phases, silica chromatography was used to purify PE, as described in Example 2. The product was eluted with a mixture of chloroform/methanol 70/30 (v/v). Approximately 257 mg of the PE were obtained after purification. The PE species mixture was also prepared from the PC species mixture from salmon liver, as described above. Structures of the PE products are shown below.

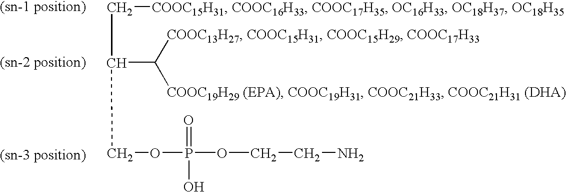

### EXAMPLE 5

### Preparation of Mixtures of sn-2-Acyl Lyso PC Species

About 200 mg of the PC species mixture extracted from the liver were dissolved in about 10 mL of a Tris buffer (100 mM; pH 6.5) in the presence of about 50 mM of calcium chloride, about 200 μl of 30% of bovine albumin solution and about 70 mg of sodium deoxycholate, and then the mixture was sonicated at about 4° C. The enzymatic reaction was started by adding about 35 mg of lipase (Rhizopus arrhizus; SIGMA, St. Louis, Mo., USA). The mixture was left to react for about 3 hours at 30° C. on a shaker. Once the reaction was complete, the vial was unloaded with about 50 mL of a mixture of chloroform/methanol 2/1 (v/v). The two separated phases were formed, and Lyso PC is in the down-phase. Lyso PC remaining in the up-phase was re-extracted with chloroform. After drying the combined two organic phases, silica chromatography (using a 150 mL-column) was used to purify Lyso PC. The product was eluted with a mixture of chloroform/methanol 50/50/2 (v/v/v). Approximately 132 mg of the product was obtained after purification. Structures and percentages of the Lyso PC species, which are tentatively analyzed by intensities of their protonated molecules of electrospray mass spectrometry, are shown below.

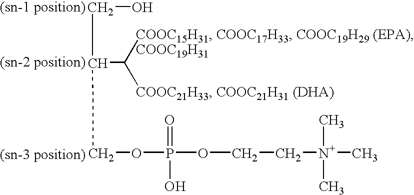

| [M + H]⁺ | Molecular Species | Shark (%) |
|---|---|---|
| 570.5 | 22:5 | 10 ± 0.35 |
| 568.5 | 22:6 | 48 ± 2.5 |
| 544.5 | 20:4 | 15 ± 0.55 |
| 542.5 | 20:5 | 14 ± 1.25 |
| 522.5 | 18:1 | 6.0 ± 0.75 |
| 496.5 | 16:0 | 6.9 ± 0.45 |

| | |
|---|---|
| Total ω-3 fatty acid species | 62 |

### EXAMPLE 6

### Preparation of Mixtures of sn-2-Acyl-Lyso PS and -Lyso PE Species

About 200 mg of transphosphatidylated-fish liver-PS were dissolved in about 10 mL of a Tris buffer (100 mM; pH 6.5) in the presence of about 50 mM of calcium chloride, about 200 μl of about 30% of bovine albumin solution and about 70 mg of sodium deoxycholate, and then the mixture was sonicated at about 4° C. After adding about 35 mg of lipase (Rhizopus arrhizus; SIGMA, St. Louis, Mo., USA), the vial was fully filled with argon. The mixture was left to react for about 3 hours at about 30° C. on a shaker. Once the reaction was complete, the vial was unloaded with about 50 mL of a mixture of chloroform/methanol 2/1 (v/v). The two phases were allowed to separate, and Lyso PS is in the down-phase. Lyso PS remaining in the up-phase was re-extracted with chloroform. After drying the combined two organic phases, silica chromatography (using a 150 mL-column) was used to purify Lyso PS. The product was eluted with a mixture of chloroform/methanol 45/55 (v/v). The Lyso PE species mixture was also prepared from transphosphatidylated-fish liver-PE as described above, and was eluted with chloroform/methanol 55/45 (v/v). Structures of the two mixtures are shown below. The molecular species were identified by negative-ion electrospray mass spectrometry.

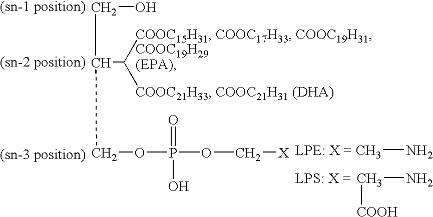

### EXAMPLE 7

### Preparation of a Mixture of 1-O-Alkyl-2-Hydroxyl-Phosphatidylcholine Species

Approximately 500 mg of dried crude phospholipids extracted from shark liver were dissolved with about 18 mL of methanol, and then about 2 mL of 5M NaOH were added. The mixture was left to react at room temperature for overnight (more than about 15 hours). The lipids in reacted solution were extracted by adding about 22 mL of methanol, about 40 mL of chloroform and about 34 mL of water. After removing the up-phase, another about 2 mL of chloroform were added, and the solution was left at about −20° C. for several hours. The clear solution was taken out, and the mixture of 1-O-alkyl-2-hydroxy-phosphatidylcholine species was purified with silica column (a 150 mL-column) as described in Example 5. The product was eluted with a mixture of chloroform/methanol/water (50/50/2). About 97 mg of the product were obtained. Structures of the mixture of 1-O-alkyl-2-hydroxy-phosphatidylcholine species are shown below.

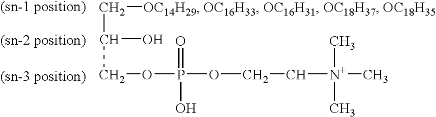

### EXAMPLE 8

### Preparation of Mixture of 1-O-Alkyl-2-DHA-Phosphatidylcholine Species

About 70 mg of 1-O-alkyl-2-hydroxyl PC species mixture, about 100 mg of DHA (SIGMA, St. Louis, Mo., USA), about 160 mg of dicyclohexylcarbodiimide (SIGMA, St. Louis, Mo., USA) and about 240 mg of 4-dimethylaminopyridine (SIGMA, St. Louis, Mo., USA) were dissolved in 3 mL of chloroform (HPLC grade). The vial was generally fully filled with argon and then put into another container that was generally fully filled with nitrogen. It was left to react at about 40° C. for about 20 hours. About one mL of chloroform was added, and the reaction mixture was then applied to a 150-mL silica column equilibrated with chloroform/methanol 95/5 (v/v). The PC product was eluted with a mixture of chloroform/methanol 50/50 (v/v). Structures (shown as below) of the product were identified by mass spectrometry as O-14:0/22:6 PC ([M+H]⁺ 764.6; 9%). O-16:1/22:6 PC ([M+H]⁺ 790.6; 18%), O-16:0/22:6 PC ([M+H]⁺ 792.6; 25%), O-18:1/22:6 PC ([M+H]⁺ 818.6; 33%) and O-18:0/22:6 PC ([M+H]⁺ 820.7; 15%).

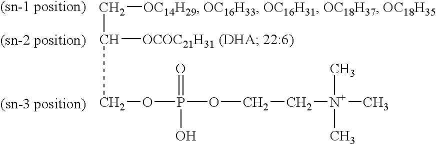

### EXAMPLE 9

### Preparation of Mixtures of 1-O-Alkyl-2-DHA-PS and -PE Species

About 1 mL of acetate buffer (0.2M; pH 5.5), containing about 40 mM of calcium chloride and about 500 mg of L-serine was prepared in a 20-mL disposable vial at about 45° C. About 15 mg of 1-O-alkyl-DHA-PC species mixture were added to the vial generally fully filled with argon. The enzymatic reaction was started by adding 1 Unit of phospholipase D (Streptomyces sp, BIOMOL, Plymouth Meeting, Pa., U.S.A). It was left to react for about 15 hours at about 40° C. on a shaker. Once the reaction was complete, the vial was unloaded with about 10 mL of a mixture of chloroform/methanol 2/1 (v/v) and 1 mL of water. The two separated phases were formed, and alkyl PS product was in the down-phase. Alkyl PS remaining in the up-phase was re-extracted with chloroform. After drying the combined two organic phases, silica chromatography (using a 150 mL-column) was used to purify the PS, as described in Example 5. The product was eluted with a mixture of chloroform/methanol (55/45).

About 1 mL of acetate buffer (0.2M; pH 5.5), containing about 40 mM of calcium chloride and about 200 mg of ethanolamine were prepared in a 20-mL disposable vial at room temperature, and then pH of the solution was adjusted to the pH of about 5.5 with glacial acetic acid. About 15 mg of 1-O-alkyl-DHA-PC species mixture were added. The vial was generally fully filled with argon. The enzymatic reaction was started by adding about 1 unit of phospholipase D (Streptomyces sp, BIOMOL, Plymouth Meeting, Pa., USA). It was left to react for about 15 hours at about 40° C. on a shaker. Once the reaction was complete, the vial was unloaded with about 10 mL of a mixture of chloroform/methanol 2/1 (v/v) and one mL of water. The two separated phases were formed, and alkyl PE is in the down-phase. Alkyl PE remaining in the up-phase was re-extracted with chloroform. After drying the combined two organic phases, silica chromatography was used to purify PE, as described in Example 5. The product was eluted with a mixture of chloroform/methanol 70/30 (v/v). The structures of the two mixtures are shown as below. The molecular species were identified by negative-ion electrospray mass spectrometry.

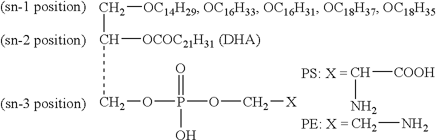

### EXAMPLE 10

### Structural Identification of Alkyl Bond in Ether Phospholipid Species from the Shark Liver, Unusual DHA-Phospholipid Species and sn-2-DHA-Lysophospholipid Species

Acid hydrolysis was performed by treating approximately 1 mg of 1-O-alkyl-2-hydroxy-PC species mixture with about 1 mL of 0.012 M hydrochloric acid in methanol and about 0.5 mL of chloroform for about 20 min at room temperature. A control sample was also made by treating approximately 1 mg of 1-O-alkyl-2-hydroxy-PC species mixture with about 1 mL of water in methanol and about 0.5 mL of chloroform for about 20 min at room temperature. After extraction of the lipids in the two solutions, the lipid species were analyzed by positive-ion electrospray mass spectrometry. It has been found that the percentage of five alkyl Lyso PC molecular species in the control sample are almost identical to that of the alkyl Lyso PC species in the samples treated with hydrochloric acid. It indicated that ether lipid fatty chains, linked to the sn-1 position, in mixtures of ether phospholipid species are 1-O-alkyl linkage, rather than 1-alk-1′-enyl linkage (plasmalogen species).

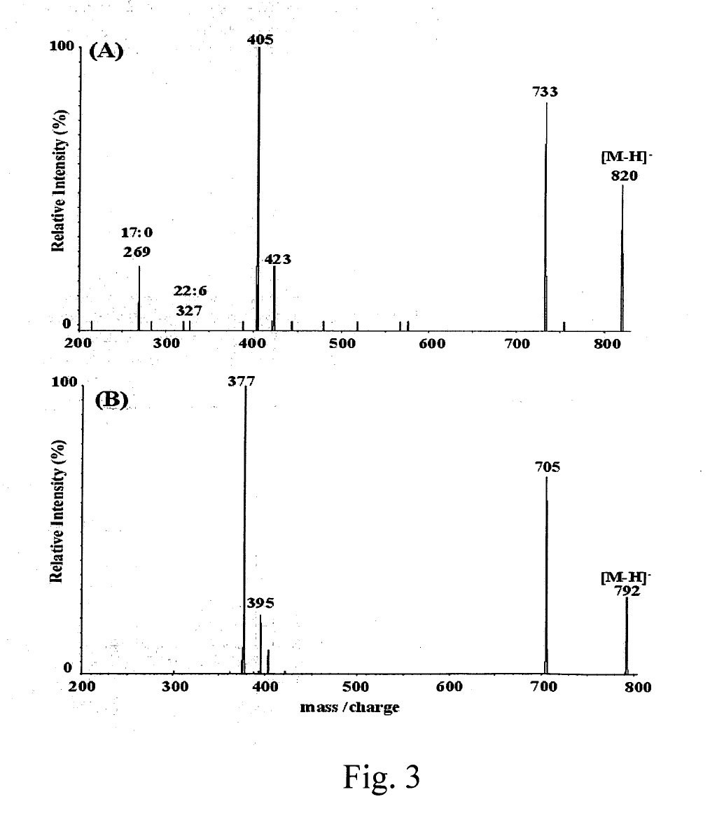

The PC molecular species from the liver of the fishes were identified by collision-induced-dissociation (CID) of deprotonated molecules of transphosphatidylated fish liver-PS because the fatty chains linked to the sn-1 and sn-2 positions in mixtures of transphosphatidylated-PS and -PE species are almost identical to those of the PC species mixtures after the transphos-phatidylation. Structures of unusual molecular species in the liver of saltwater fishes, such as sn-1-17:0-sn-2-DHA PC species and 1-O-16:0-sn-2-DHA PC species, have been identified by electrospray ionization CID of deprotonated molecules of transphosphatidylated fish liver-PS species using published methods. Refer to FIGS. 3(A) and 3(B), where FIG. 3 (A) is the CID product ion spectra of deprotonated molecules of sn-1-17:0-sn-2-DHA PS species; and FIG. 3(B) is the CID product ion spectra of deprotonated molecules of 1-O-16:0-sn-2-DHA PS species. The gas-phase ion chemistry of the phospholipid species have been described in details in the papers [Chen, Tandem mass spectrometric approach for analyzing the molecular species aminophospholipids, Lipids, 28, 85 (1997); Chen et, al. Biomed. Mass Spectrom. 21, 655 (1992); Chen and Claeys, Characterization of ω-3-docosahexaenoic acid-containing molecular species of phospholipids in rainbow trout liver, J. Agr. Food Chem. 42, 3210 (1996)].

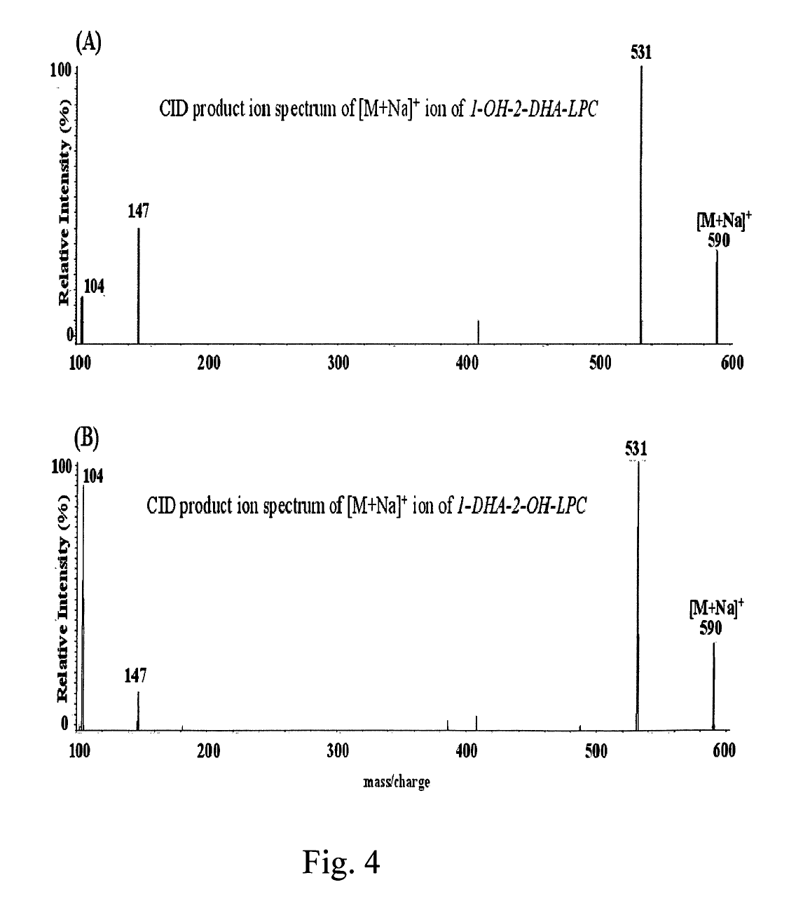

The difference in chemical structures of sn-1-DHA-sn-2-OH-Lyso PC and sn-1-OH-sn-2-DHA-Lyso PC in the species mixtures (from shark liver) were identified by CID of [M+Na]⁺ adduct ions of the species, based on the published method [Han and Gross, Structural determination of lysophospholipid regioisomers by electrospray ionization tandem mass spectrometry, J. Am. Chem. Soc., 118, 451 (1996)]. The peak intensity ratio of product ions 147/104, generated by CID of sodiated sn-1-OH-sn-2-DHA Lyso PC species (see FIG. 4(A) which is a CID product ion mass spectra of sn-1-OH-sn-2-DHA LPC), is greater than that of product ions 147/104, generated by electrospray ionization CID of sodiated sn-1-DHA-sn-2-OH Lyso PC species (see FIG. 4(B) which is a CID product ion mass spectra of sn-1-DHA-sn-2-OH LPC).

A PE SCIEX (Toronto, Canada) API 3000 triple-quadrupole tandem mass spectrometer equipped is performed using a triple-quadrupole tandem mass spectrometer equipped with a Turbo Ionspray interface of electrospray ionization. For the analyses of Lyso PC isomers, all samples are dissolved in 1:1:0.01 chloroform/methanol/NaCl (1M), and infused directly into the mass spectrometer using a syringe pump as described in the reference paper; for the analyses of PC, PE and PC mixtures, all sample are dissolved in 1:1 chloroform/methanol, and infused directly into the mass spectrometer, as described in the reference papers.

It should be noted that the terms “first”, “second”, and “third”, and the like may be used herein to modify elements performing similar and/or analogous functions. These modifiers do not imply a spatial, sequential, or hierarchical order to the modified elements unless specifically stated.

While the disclosure has been described with reference to several embodiments, it will be understood by those skilled in the art that various changes may be made and equivalents may be substituted for elements thereof without departing from the scope of the disclosure. In addition, many modifications may be made to adapt a particular situation or material to the teachings of the disclosure without departing from the essential scope thereof. Therefore, it is intended that the disclosure not be limited to the particular embodiments disclosed as the best mode contemplated for carrying out this disclosure, but that the disclosure will include all embodiments falling within the scope of the appended claims.

What is claimed is:

1. A mixture of natural phosphatidylcholine species as shown in Formula 1: 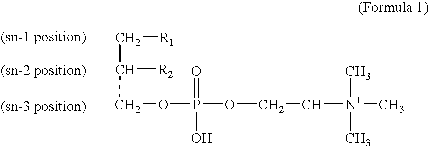
wherein R₁ is a mixture of acyl and alkyl fatty chains, linked to the sn-1 position, selected from the group consisting of COOC₁₅H₃₁ (acyl fatty chain; palmitic acid; 16:0); COOC₁₆H₃₃ (acyl fatty chain; margaric acid; 17:0); COOC₁₇H₃₅ (acyl fatty chain; stearic acid; 18:0); OC₁₆H₃₃ (alkyl fatty chain; O-16:0); OC₁₈H₃₇ (alkyl fatty chain; O-18:0); and OC₁₈H₃₅ (alkyl fatty chain; O-18:1); and
wherein R₂ consists of a mixture of acyl fatty chains, linked to the sn-2 position, and selected from the group consisting of COOC₁₃H₂₇ (acyl fatty chain; myristic acid; 14:0); COOC₁₅H₃₁ (acyl fatty chain; palmitic acid; 16:0); COOC₁₅H₂₉ (acyl fatty chain; palmitoleic acid; 16:1); COOC₁₇H₃₃ (acyl fatty chain, oleic acid; 18:1); COOC₁₉H₃₁ (acyl fatty chain; arachidonic acid; 20:4); COOC₁₉H₂₉ (acyl fatty chain; eicosapentaenoic acid; 20:5 (ω-3)); COOC₂₁H₃₃ (acyl fatty chain; docosapentanoic acid; 22:5); and COOC₂₁H₃₁ (acyl fatty chain; docosahexaenoic acid; 22:6 (ω-3)).

2. A mixture of natural lysophosphatidylcholine species as shown in Formula 2: 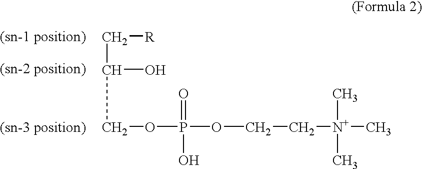
wherein R is a mixture of acyl and alkyl fatty chains, linked to the sn-1 position, and selected from the group consisting of COOC₁₃H₂₇ (acyl fatty chain; myristic acid; 14:0); COOC₁₅H₃₁ (acyl fatty chain; palmitic acid; 16:0); COOC₁₅H₂₉ (acyl fatty chain; palmitoleic acid; 16:1); COOC₁₇H₃₅ (acyl fatty chain: stearic acid; 18:0); COOC₁₇H₃₃ (acyl fatty chain; oleic acid; 18:1); COOC₁₇H₃₁ (acyl fatty chain; linoleic acid; 18:2); COOC₁₉H₃₁ (acyl fatty chain; arachidonic acid; 20:4); COOC₁₉H₂₉ (acyl fatty chain; eicosapentaenoic acid; 20:5 (ω-3)); COOC₂H₃₁ (acyl fatty chain; docosahexaenoic acid; 22:6 (ω-3)); OC₁₄H₂₉ (alkyl fatty chain; O-14:0); OC₁₆H₃₃ (alkyl fatty chain; O-16:0); OC₁₆H₃₁ (alkyl fatty chain; O-16:1); OC₁₈H₃₇ (alkyl fatty chain; O-18:0); and OC₁₈H₃₅ (alkyl fatty chain; O-18:1).

3. The mixture of claim 1 wherein said the mixture is extracted from the liver of saltwater fishes.

4. The mixture of claim 2 wherein said the mixture is extracted from the liver of saltwater fishes.

5. The mixture of claim 3 wherein said saltwater fishes are selected from the group consisting of shark, tuna, salmon and common dolphin (coryphaena hippurus).

6. The mixture of claim 4 wherein said saltwater fishes are selected from the group consisting of shark, tuna, salmon and common dolphin (coryphaena hippurus).

7. A mixture of phosphatidylserine species as shown in Formula 3: 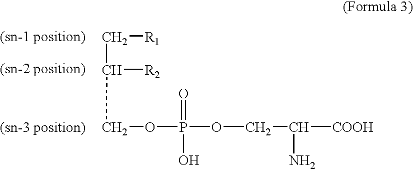
wherein R₁ is a mixture of acyl and alkyl fatty chains, linked to the sn-1 position, selected from the group consisting of COOC₁₅H₃₁ (acyl fatty chain; palmitic acid; 16:0); COOC₁₆H₃₃ (acyl fatty chain; margaric acid; 17:0); COOC₁₇H₃₅ (acyl fatty chain; stearic acid; 18:0); OC₁₆H₃₃ (alkyl fatty chain; O-16:0); OC₁₈H₃₇, (alkyl fatty chain; O-18:0); and OC₁₈H₃₅ (alkyl fatty chain; O-18:1); and
wherein R₂ consists of a mixture of acyl fatty chains, linked to the sn-2 position, and selected from the group consisting of COOC₁₃H₂₇ (acyl fatty chain; myristic acid; 14:0); COOC₁₅H₃₁ (acyl fatty chain; palmitic acid; 16:0); COOC₁₅H₂₉ (acyl fatty chain; palmitoleic acid; 16:1); COOC₁₇H₃₃ (acyl fatty chain; oleic acid; 18:1); COOC₁₉H₃₁ (acyl fatty chain; arachidonic acid; 20:4); COOC₁₉H₂₉ (acyl fatty chain; eicosapentaenoic acid; 20:5 (ω-3)); COOC₂₁H₃₃ (acyl fatty chain; docosapentanoic acid; 22:5); and COOC₂₁H₃₁ (acyl fatty chain; docosahexaenoic acid; 22:6 (ω-3)).

8. A mixture of phosphatidylethanolamine species as shown in Formula 4: 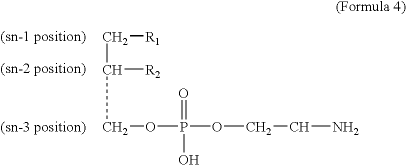
wherein R₁ is a mixture of acyl and alkyl fatty chains, linked to the sn-1 position, selected from the group consisting of COOC₁₅H₃₁ (acyl fatty chain; palmitic acid; 16:0); COOC₁₆H₃₃ (acyl fatty chain; margaric acid; 17:0); COOC₁₇H₃₅ (acyl fatty chain; stearic acid; 18:0); OC₁₆H₃₃ (alkyl fatty chain; O-16:0); OC₁₈H₃₇, (alkyl fatty chain; O-18:0); and OC₁₈H₃₅ (alkyl fatty chain; O-18:1); and
wherein R₂ consists of a mixture of acyl fatty chains, linked to the sn-2 position, and selected from the group consisting of COOC₁₃H₂₇ (acyl fatty chain; myristic acid; 14:0); COOC₁₅H₃₁ (acyl fatty chain; palmitic acid; 16:0); COOC₁₅H₂₉ (acyl fatty chain; palmitoleic acid; 16:1); COOC₁₇H₃₃ (acyl fatty chain; oleic acid; 18:1); COOC₁₉H₃₁ (acyl fatty chain; arachidonic acid; 20:4); COOC₁₉H₂₉ (acyl fatty chain; eicosapentaenoic acid; 20:5 (ω-3)); COOC₂₁H₃₃ (acyl fatty chain; docosapentanoic acid; 22:5); and COOC₂₁H₃₁ (acyl fatty chain; docosahexaenoic acid; 22:6 (ω-3)).

9. The mixture of claim 7 wherein said the mixture of phosphatidylserine species is prepared from a mixture of natural phosphatidylcholine species extracted from the liver of saltwater fishes by the transphosphatidylation.

10. The mixture of claim 8 wherein said the mixture of phosphatidylethanolamine species is prepared from a mixture of natural phosphatidylcholine species extracted from the liver of saltwater fishes by the transphosphatidylation.

11. A mixture of 1-hydroxy-2-acyl-phosphatidylcholine species as shown in Formula 5. 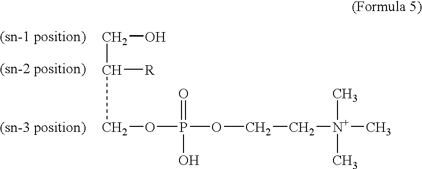
wherein R is a mixture of acyl fatty chains linked to the sn-2 position, selected from the group consisting of COOC₁₅H₃₁ (acyl fatty chain; palmitic acid; 16:0); COOC₁₇H₃₃ (acyl fatty chain; oleic acid; 18:1); COOC₁₉H₃₁ (acyl fatty chain; arachidonic acid; 20:4); —COOC₁₉H₂₉ (acyl fatty chain; eicosapentaenoic acid; 20:5 (ω-3)); COOC₂₁H₃₃ (acyl flaw chain; docosapentanoic acid; 22:5); and COOC₂₁H₃₁ (acyl fatty chain; docosahexaenoic acid; 22:6 (ω-3)).

12. The mixture of claim 11 wherein said the mixture of 1-hydroxy-2-acyl-phosphatidylcholine molecular species is prepared from a mixture of natural phosphatidylcholine species extracted from the liver of saltwater fishes by the lipase hydrolysis.

13. A mixture of 1-hydroxy-2-acyl-phosphatidylserine molecular species as shown in Formula 6: 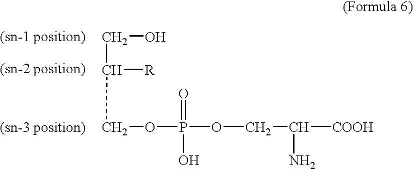
wherein R is a mixture of acyl fatty chains linked to the sn-2 position, selected from the group consisting of COOC₁₅H₃₁ (acyl fatty chain; palmitic acid; 16:0); COOC₁₇H₃₃ (acyl fatty chain; oleic acid; 18:1); COOC₁₉H₃₁ (acyl fatty chain; arachidonic acid; 20:4); COOC₁₉H₂₉ (acyl fatty chain; eicosapentaenoic acid; 20:5 (ω-3)); COOC₂₁H₃₃ (acyl fatty chain; docosapentanoic acid; 22:5); and COOC₂₁H₃₁ (acyl fatty chain; docosahexaenoic acid 22:6 (ω-3)).

14. The mixture of claim 13 wherein said the mixture of 1-hydroxy-2-acyl-phosphatidylserine is prepared from a mixture of phosphatidylserine species as shown in Formula 3 by the lipase hydrolysis: 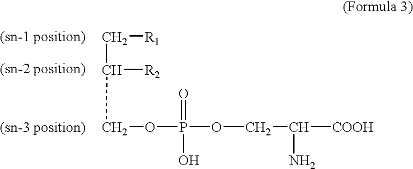
wherein R₁ is a mixture of acyl and alkyl fatty chains, linked to the sn-1 position, and selected from the group consisting of COOC₁₅H₃₁ (acyl fatty chain; palmitic acid; 16:0); COOC₁₆H₃₃ (acyl fatty chain; margaric acid; 17:0); COOC₁₇H₃₅ (acyl fatty chain; stearic acid; 18:0); OC₁₆H₃₃ (alkyl fatty chain; O-16:0); OC₁₈H₃₇ (acyl fatty chain; O-18:0); and OC₁₈H₃₅ (alkyl fatty chain; O-18:1); and
wherein R₂ consists of a mixture of acyl fatty chains linked to the sn-2 position, and selected from the group consisting of COOC₁₃H₂₇ (acyl fatty chain; myristic acid; 14:0); COOC₁₅H₃₁ (acyl fatty chain; palmitic acid; 16:0); COOC₁₅H₂₉ (acyl fatty chain; palmitoleic acid; 16:1); COOC₁₇H₃₃ (acyl fatty chain; oleic acid; 18:1); COOC₁₉H₃₁ (acyl fatty chain; arachidonic acid; 20:4); COOC₁₉H₂₉ (acyl fatty chain; eicosapentaenoic acid; 20:5 (ω-3)); COOC₂₁H₃₃ (acyl fatty chain; docosapentanoic acid: 22:5); and COOC₂₁H₁₃, (acyl fatty chain; docosahexaenoic acid; 22:6 (ω-3)).

15. A mixture of 1-hydroxy-2-acyl-phosphatidylethanolamine molecular species as shown in Formula 7: 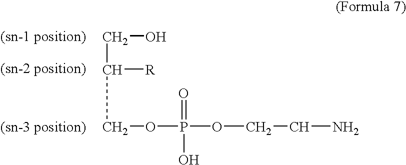
wherein R is a mixture of acyl fatty chains linked to the sn-2 position, selected from the group consisting of COOC₁₅H₃₁ (acyl fatty chain; palmitic acid; 16:0); COOC₁₇H₃₃ (acyl fatty chain; oleic acid 18:1); COOC₁₉H₃₁ (acyl fatty chain; arachidonic acid; 20:4); COOC₁₉H₂₉ (acyl fatty chain; eicosapentaenoic acid 90:5 (ω-3)); COOC₂₁H₃₃ (acyl fatty chain; docosapentanoic acid; 22:5); and COOC₂₁H₃₁ (acyl fatty chain; docosahexaenoic acid; 22:6 (ω-3)).

16. The mixture of claim 15 wherein said the mixture of 1-hydroxy-2-acyl-phosphatidyl-ethanolamine species is prepared from a mixture of phosphatidylethanolamine species as shown in formula 4 by the lipase hydrolysis: 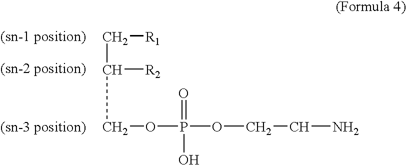
wherein R₁ is a mixture of acyl and alkyl fatty chains, linked to the sn-1 position, and selected from the group consisting of COOC₁₅H₃₁ (acyl fatty chain; palmitic acid; 16:0); COOC₁₆H₃₃ (acyl fatty chain; margaric acid; 17:0); COOC₁₇H₃₅ (acyl fatty chain; stearic acid; 18:0); OC₁₆H₃₃ (alkyl fatty chain; O-16:0); OC₁₈H₃₇ (acyl fatty chain; O-18:0); and OC₁₈H₃₅ (alkyl fatty chain; O-18:1); and
wherein R₂ consists of a mixture of acyl fatty chains, linked to the sn-2 position, and selected from the group consisting of COOC₁₃H₂₇ (acyl fatty chain; myristic acid; 14:0); COOC₁₅H₃₁ (acyl fatty chain; palmitic acid; 16:0); COOC₁₃H₂₉ (acyl fatty chain; palmitoleic acid; 16:1); COOC₁₇H₃₃ (acyl fatty chain; oleic acid; 18:1); COOC₁₉H₃₁ (acyl fatty chain; arachidonic acid; 20:4); COOC₁₉H₂₉ (acyl fatty chain; eicosapentaenoic acid; 20:5 (ω-3)); COOC₂₁H₃₃ (acyl fatty chain; docosapentanoic acid: 22:5); and COOC₂₁H₃₁ (acyl fatty chain; docosahexaenoic acid; 22:6 (ω-3)).

17. A mixture of 1-O-alkyl-2-hydroxy phosphatidylcholine species as Formula 8: 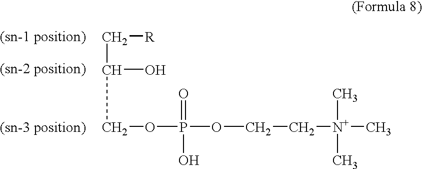
wherein R is a mixture of alkyl fatty chains linked to the sn-1 position, selected from the group consisting of OC₁₄H₂₉ (alkyl fatty chain; O-14:0); OC₁₆H₃₃ (alkyl fatty chain; O-16:0); OC₁₆H₃₁ (alkyl fatty chain; O-16:1); OC₁₈H₃₇ (alkyl fatty chain; O-18:0); and OC₁₈H₃₅ (alkyl fatty chain; O-18:1).

18. The mixture of claim 17 wherein said the mixture of 1-O-alkyl-2-hydroxy phosphatidylcholine species is prepared from a mixture of natural phosphatidylcholine species extracted from the liver of saltwater fishes.

19. The mixture of claim 17 wherein said the mixture of 1-O-alkyl-2-hydroxy phosphatidylcholine species is prepared from a mixture of natural lysophosphatidylcholine species extracted from the liver of saltwater fishes

20. A mixture of 1-O-alkyl-2-docosaheaxnoyl phosphatidylcholine species as shown in Formula 9: 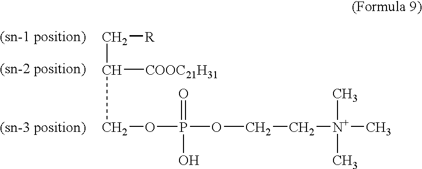
wherein R is a mixture of alkyl fatty chains linked to the sn-1 position, selected from the group consisting of OC₁₄H₂₉ (alkyl fatty chain; O-14:0); OC₁₆H₃₃ (alkyl fatty chain; O-16:0); OC₁₆H₃₁ (alkyl fatty chain; O-16:1); OC₁₈H₃₇ (alkyl fatty chain; O-18:0); and OC₁₈H₃₅ (alkyl fatty chain; O-18:1).

21. The mixture of claim 20 wherein said a mixture of 1-O-alkyl-2-docosahexaenoyl phosphatidylcholine molecular species is prepared from a mixture of 1-O-alkyl-2-hydroxy phosphatidylcholine species as shown as formula 8: 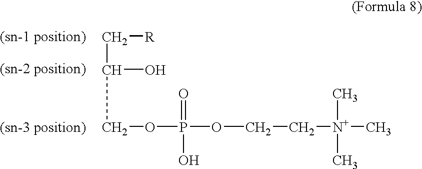
wherein R is a mixture of alkyl fatty chains linked to the sn-1 position, selected from the group consisting of OC₁₄H₂₉ (alkyl fatty chain; O-14:0); OC₁₆H₃₃ (alkyl fatty chain; 0-16:0); OC₁₆H₃₁ (alkyl fatty chain; O-16:1); OC₁₈H₃₇ (alkyl fatty chain; O-18:0); and OC₁₈H₃₅ (alkyl fatty chain; O-18:1).

22. A mixture of 1-O-alkyl-2-docosahexaenoyl phosphatidylserine species as shown in Formula 10: 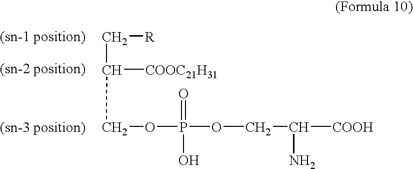
wherein R is a mixture of alkyl fatty chains linked to the sn-1 position, selected from the group consisting of OC₁₄H₂₉ (alkyl fatty chain; O-14:0); OC₁₆H₃₃ (alkyl fatty chain; O-16:0); OC₁₆H₃₁ (alkyl fatty chain; O-16:1); OC₁₈H₃₇ (alkyl fatty chain; O-18:0); and OC₁₈H₃₅ (alkyl fatty chain; O-18:1.

23. The mixture of claim 22 wherein the mixture of 1-O-alkyl-2-docosahexaenoyl phosphatidylserine species is prepared from a mixture of 1-O-alkyl-2-docosahexaenoyl phosphatidylcholine molecular species as shown in Formula 9 by the transphosphatidylation: 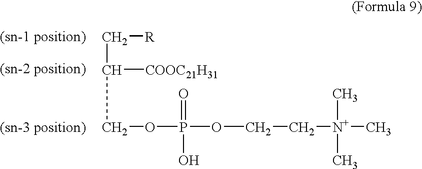
wherein R is a mixture of alkyl fatty chains linked to the sn-1 position, selected from the group consisting of OC₁₄H₂₉ (alkyl fatty chain; O-14:0); OC₁₆H₃₃ (alkyl fatty chain; O-16:0); OC₁₆H₃₁ (alkyl fatty chain; O-16:1); OC₁₈H₃₇ (alkyl fatty chain; O-18:0); and OC₁₈H₃₅ (alkyl fatty chain; O-18:1).

24. A mixture of 1-O-alkyl-2-docosahexaenoyl phosphatidylethanolamine species as shown in Formula 11. 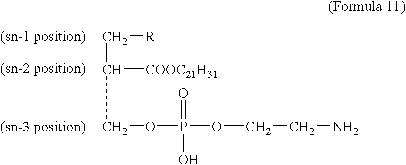
wherein R is a mixture of alkyl fatty chains linked to the sn-1 position, selected from the group consisting of OC₁₄H₂₉ (alkyl fatty chain; O-14:0); OC₁₆H₃₃ (alkyl fatty chain; O-16:0); OC₁₆H₃₁ (alkyl fatty chain; O-16:1); OC₁₈H₃₇ (alkyl fatty chain; O-18:0); and OC₁₈H₃₅ (alkyl fatty chain; O-18:1).

25. The mixture of claim 24 wherein the mixture of 1-O-alkyl-2-docosahexaenoyl phosphatidylethanolamine species is prepared from a mixture of 1-O-alkyl-2-docosahexaenoyl phosphatidylcholine molecular species as shown in Formula 9 by the transphosphatidylation: 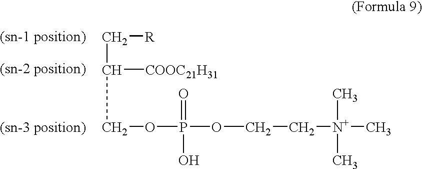
wherein R is a mixture of alkyl fatty chains linked to the sn-1 position, selected from the group consisting of OC₁₄H₂₉ (alkyl fatty chain; O-14:0); OC₁₆H₃₃ alkyl fatty chain; O-16:0); OC₁₆H₃₁ (alkyl fatty chain; O-16:1); OC₁₈H₃₇ (alkyl fatty chain; O-18:0); and OC₁₈H₃₅ (alkyl fatty chain; O-18:1).

26. A method of treating a mammal comprising:
administrating a therapeutically effective amount of the mixture of claim 1 to a mammal under conditions selected from the group consisting of conditions effective to recovery a quantity of neurotrophin receptors in the mammal's brain; conditions effective to recovery functionality of neurotrophins in the brain of the mammal; conditions effective to recovery the functionality of choline acetyltransferase and tyrosine hydroxylase in a mammal's brain; conditions effective to alleviate and treat neurodegenerative diseases; conditions effective to reduce the deleterious effects of normal brain aging; under conditions effective to treat Meniere's disease; conditions effective to treat depression; conditions effective to treat psychological stress; and under conditions effective to treat multiple sclerosis.

27. The method of claim 26, wherein the neurotrophin receptors are selected from the group consisting of p75 pan neurotrophin receptor (p75^{NTR}); and Trk family of tyrosine kinase receptors including Trk, TrkB and TrkC.

28. The method of claim 26, wherein the neurotrophins are selected from the group consisting of nerve growth factor (NGF), brain derived neurotrophic factor (BDNF) neurotrophin 3 (NT-3) and neurotrophin 4 and 5 (NT-4/5).

29. The method of claim 26, wherein said neurodegenerative diseases is selected from the group consisting of Alzheimer's disease, Huntington disease and Parkinson's disease.

30. A method of treating a mammal comprising:
administrating a therapeutically effective amount of the mixture of claim 2 to a mammal under conditions selected from the group consisting of conditions effective to recovery a quantity of neurotrophin receptors in the mammal's brain; conditions effective to recovery functionality of neurotrophins in the brain of the mammal; conditions effective to recovery the functionality of choline acetyltransferase and tyrosine hydroxylase in a mammal's brain; conditions effective to alleviate and treat neurodegenerative diseases; conditions effective to reduce the deleterious effects of normal brain aging; under conditions effective to treat Meniere's disease; conditions effective to treat depression; conditions effective to treat psychological stress; and under conditions effective to treat multiple sclerosis.

31. The method of claim 30, wherein the neurotrophin receptors are selected from the group consisting of p75 pan neurotrophin receptor (p75^{NTR}); and Trk family of tyrosine kinase receptors including TrkA, TrkB and TrkC.

32. The method of claim 30, wherein the neurotrophins are selected from the group consisting of nerve growth factor (NGF), brain derived neurotrophic factor (BDNF), neurotrophin 3 (NT-3) and neurotrophin 4 and 5 (NT-45).

33. The method of claim 30, wherein said neurodegenerative diseases is selected from the group consisting of Alzheimer's disease, Huntington disease and Parkinson's disease.

34. A method of treating a mammal comprising:
administrating a therapeutically effective amount of the mixture of claim 7 to a mammal under conditions selected from the group consisting of conditions effective to recovery a quantity of neurotrophin receptors in the mammal's brain; conditions effective to recovery functionality of neurotrophins in the brain of the mammal; conditions effective to recovery the functionality of choline acetyltransferase and tyrosine hydroxylase in a mammal's brain; conditions effective to alleviate and treat neurodegenerative diseases; conditions effective to reduce the deleterious effects of normal brain aging; under conditions effective to treat Meniere's disease; conditions effective to treat depression; conditions effective to treat psychological stress; and under conditions effective to treat multiple sclerosis.

35. The method of claim 34, wherein the neurotrophin receptors are selected from the group consisting of p75 pan neurotrophin receptor (p75^{NTR}); and Trk family of tyrosine kinase receptors including TrkA, TrkB and TrkC.

36. The method of claim 343 wherein the neurotrophins are selected from the group consisting of nerve growth factor (NGF), brain derived neurotrophic factor (BDNF), neurotrophin 3 (NT-3) and neurotrophin 4 and 5 (—NT-4/5).

37. The method of claim 34, wherein said neurodegenerative diseases is selected from the group consisting of Alzheimer's disease, Huntington disease and Parkinson's disease.
